(19) United States
(12) Patent Application Publication
(10) Pub. No.: US 2015/0045537 A1
(43) Pub. Date: Feb. 12, 2015

(54) EXTRACELLULAR RELEASE OF LIPIDS BY PHOTOSYNTHETIC CELLS

(71) Applicant: Massachusetts Institute of Technology, Cambridge, MA (US)

(72) Inventors: Sallie Watson Chisholm, Watertown, MA (US); Steven James Biller, Watertown, MA (US); Anne Williford Thompson, Santa Cruz, CA (US)

(73) Assignee: Massachusetts Institute of Technology, Cambridger, MA (US)

(21) Appl. No.: 14/385,519

(22) PCT Filed: Mar. 12, 2013
(86) PCT No.: PCT/US13/30516

§ 371 Date: Sep. 16, 2014

Related U.S. Application Data
(60) Provisional application No. 61/611,664, filed on Mar. 16, 2012.

(51) Int. Cl. C12N 1/12; C11B 3/00; C07K 14/405

(52) U.S. Cl. CPC C12N 1/12; C07K 14/405; C11B 3/006
USPC 530/350; 435/134; 435/257.1; 554/1; 435/71.2; 435/91.1

(57) ABSTRACT

The invention relates to methods for harvesting macromolecules such as lipids and proteins by culturing in growth medium a photo synthetic cyanobacteria that secretes vesicles into the growth medium, and separating the secreted vesicles from the cyanobacteria and/or from the growth medium.

### RELATED APPLICATIONS

This application claims the benefit under 35 U.S.C. §119(e) of U.S. Provisional Application Ser. No. 61/611,664, filed on Mar. 16, 2012, the entire contents of which is incorporated by reference herein in its entirety.

### GOVERNMENT INTEREST

This invention was made with Government support under Grant No. OCE0425602 awarded by the National Science Foundation, and under Grant No. DE-FG02-08ER64516 awarded by the U.S. Department of Energy. The Government has certain rights in this invention.

### FIELD OF THE INVENTION

The invention relates to photosynthetic production and harvesting of vesicles comprising macromolecules such as lipids, proteins and nucleic acids.

### BACKGROUND OF THE INVENTION

Photosynthetic microorganisms hold great promise as a source of renewable biofuels that could replace traditional fossil fuels. Microbes are potentially more attractive for this purpose than plants for many reasons, including high productivity, the ability to use non-agriculturally productive land, and the fact that they are a non-food resource (16). Over the last three decades, most of the research in this field has focused on the use of microalgae to produce lipids (such as triacylglycerols) that can be converted into biodiesel and other energy-rich fuels.

Despite the great potential for microbially derived biofuels, commercially viable production has not yet been achieved. One problem with the use of algae is that high lipid content is only achieved when grown under environmentally stressful conditions that can be costly and difficult to maintain (6). Microalgal lipids accumulate within the cell, and purifying the high-value lipids away from the rest of the biomass also has proven to be a significant challenge. Typically, cells are collected by centrifugation or flocculation and then subjected to energetically-costly processes to extract the lipids using solvents or mechanical methods (2, 5, 7).

### SUMMARY OF INVENTION

Described herein is a novel mechanism through which macromolecules such as high-energy lipid compounds, or proteins, can be harvested from some of the world's smallest and most efficient photosynthetic organisms, such as the marine cyanobacterium Prochlorococcus. It was surprisingly discovered that, in contrast to intracellular lipid accumulation by algae, Prochlorococcus naturally releases lipids into growth medium in the form of small vesicles. This is the first description of the production of such lipid-containing vesicles by a photosynthetic microorganism. Significantly, the mechanisms behind this cellular capability facilitate a biofuel production scheme wherein the small lipid-containing vesicles are collected from the medium without the need to harvest cells or separate lipids away from the bulk cellular biomass.

According to one aspect of the invention, methods for harvesting lipids are provided. The methods include culturing in growth medium a photosynthetic cyanobacteria that secretes lipid-containing vesicles into the growth medium; and separating the secreted lipid-containing vesicles from the cyanobacteria and/or from the growth medium.

In some embodiments, the methods further include collecting the lipid-containing vesicles.

In some embodiments, the photosynthetic cyanobacteria are of the genus Prochlorococcus, Synechococcus or Synechocystis.

In some embodiments, the photosynthetic cyanobacteria are cultured within a bioreactor or open pond system.

In some embodiments, a carbon source is not added to the growth medium.

In some embodiments, the cyanobacteria are co-cultured with heterotrophic bacteria.

In some embodiments, lipid-containing vesicles are produced at a concentration of at least 10⁷ ml⁻¹ in the growth medium.

In some embodiments, the cyanobacterial cell is not lysed during separation of the secreted lipid-containing vesicles from the cyanobacteria.

In some embodiments, the photosynthetic cyanobacteria are exposed to constant light flux or to a light-dark cycle.

In some embodiments, the lipid-containing vesicles are purified by filtration, density gradient centrifugation or binding to an affinity column/resin.

In some embodiments, a population of photosynthetic cyanobacteria is kept within a continual growth state and lipid-containing vesicles are continually separated from the cyanobacteria and/or from the growth medium.

In some embodiments, at least 0.1 mg lipid L⁻¹ are produced.

According to another aspect of the invention, a cell culture produced by any one of the foregoing methods is provided.

According to another aspect of the invention, compositions include isolated lipid-containing vesicles of photosynthetic cyanobacteria.

In some embodiments, the lipid-containing vesicles in the compositions are present at a concentration of at least 10⁷ ml⁻¹.

In some embodiments, the lipid-containing vesicles in the compositions are substantially free of cyanobacterial cells.

In some embodiments, the lipid-containing vesicles in the compositions are purified by filtration, density gradient centrifugation or binding to an affinity column/resin.

Further aspects of the invention relate to methods for harvesting proteins, including: culturing in growth medium a photosynthetic cyanobacterium that secretes protein-containing vesicles into the growth medium; and separating the secreted protein-containing vesicles from the cyanobacteria and/or from the growth medium.

In some embodiments, methods further include collecting the protein-containing vesicles. In certain embodiments, the photosynthetic cyanobacteria are of the genus Prochlorococcus, Synechococcus or Synechocystis. In some embodiments, the photosynthetic cyanobacteria are cultured within a bioreactor or open pond system.

In some embodiments, a carbon source is not added to the growth medium. In some embodiments, the cyanobacteria are co-cultured with a heterotrophic bacteria. In certain embodiments, protein-containing vesicles are produced at a concentration of at least 10⁷ ml⁻¹ in the growth medium. In some embodiments, the cyanobacterial cell is not lysed during separation of the secreted lipid-containing vesicles from the cyanobacteria. In some embodiments, the photosynthetic cyanobacteria are exposed to constant light flux or to a light-dark cycle. In some embodiments, the protein-containing vesicles are purified by filtration, density gradient centrifugation or binding to an affinity column/resin. In some embodiments, a population of photosynthetic cyanobacteria is kept within a continual growth state and wherein lipid-containing vesicles are continually separated from the cyanobacteria and/or from the growth medium. In certain embodiments, at least 0.1 mg protein L⁻¹ are produced.

According to another aspect of the invention, a cell culture produced by any one of the foregoing methods is provided.

According to another aspect of the invention, compositions are provided comprising isolated protein-containing vesicles of photosynthetic cyanobacteria.

In some embodiments, the protein-containing vesicles in the composition are present at a concentration of at least 10⁷ ml⁻¹. In some embodiments, the protein-containing vesicles in the composition are substantially free of cyanobacterial cells. In some embodiments, the protein-containing vesicles in the composition are purified by filtration, density gradient centrifugation or binding to an affinity column/resin.

Further aspects of the invention relate to methods for harvesting photosynthetically derived macromolecules, including: concentrating a solution containing photosynthetic cyanobacteria that secrete vesicles containing photosynthetically derived macromolecules into the solution; and separating the secreted vesicles containing photosynthetically derived macromolecules from the cyanobacteria and/or from the solution. In some embodiments, the macromolecule is a protein, a nucleic acid or a lipid.

These and other aspects of the invention, as well as various embodiments thereof, will become more apparent in reference to the drawings and detailed description of the invention.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF DRAWINGS

The accompanying drawings are not intended to be drawn to scale. For purposes of clarity, not every component may be labeled in every drawing. In the drawings:

FIG. 1 shows images of membrane vesicles produced by Prochlorococcus MIT9313ax. FIG. 1A shows a scanning electron microscope image of a late exponential phase culture. Samples were collected by gently filtering culture onto a 0.1 μm filter to improve recovery of the small vesicles by avoiding centrifugation steps. FIG. 1B shows that structures bound by a single membrane layer can be seen in thin-section transmission electron microscope images of Prochlorococcus MED4ax cell-free supernatant.

FIG. 2 depicts laboratory-scale production of purified lipid vesicles. Shown is a representative process described herein for purifying vesicles (small white circles) away from cells (large green circles) and other components of the media (triangles and diamonds). Cells were removed from up to 20 L of culture using a 0.2 μm capsule filter. The supernatant containing mostly vesicles was then concentrated using a tangential flow filter, collected by ultracentrifugation, and further purified over an Optiprep density gradient. The final sample was highly enriched for lipid vesicles, as confirmed by transmission electron microscopy (lower right; scale bar: 100 nm).

FIG. 3 shows enriched purified vesicles from Prochlorococcus MED4ax. FIG. 3A shows a silver-stained gel of 0-45% Optiprep gradient fractions (spun for 6 hours at approximately 100,000×g; 1=lowest density, 9=highest density) demonstrating that specific protein bands associated with vesicles migrate to specific densities. FIG. 3B shows a negative-stain transmission electron microscope image of the purified gradient fractions indicated, showing that they contain vesicles.

FIG. 4 reveals that vesicles can serve as a carbon source for marine heterotrophs. Growth (measured as OD₆₀₀) of a marine Alteromonas strain was followed in a BioTek plate reader for ˜36 hours. The heterotroph was able to grow in Pro99 only when amended with specific organic carbon sources (a) or purified Prochlorococcus vesicle material (b); it was unable to grow in seawater alone (purple) or seawater amended with a mock purification (c).

FIG. 5 demonstrates that membrane vesicles are found in cell-free Prochlorococcus supernatant. Vesicles from Prochlorococcus strain MED4ax were purified as described in FIG. 2 and visualized using (A) negative-stain transmission electron microscopy and (B) thin-section transmission electron microscopy. (C) Depicts the size distribution of vesicles obtained from MED4ax, as measured by the NanoSight instrument.

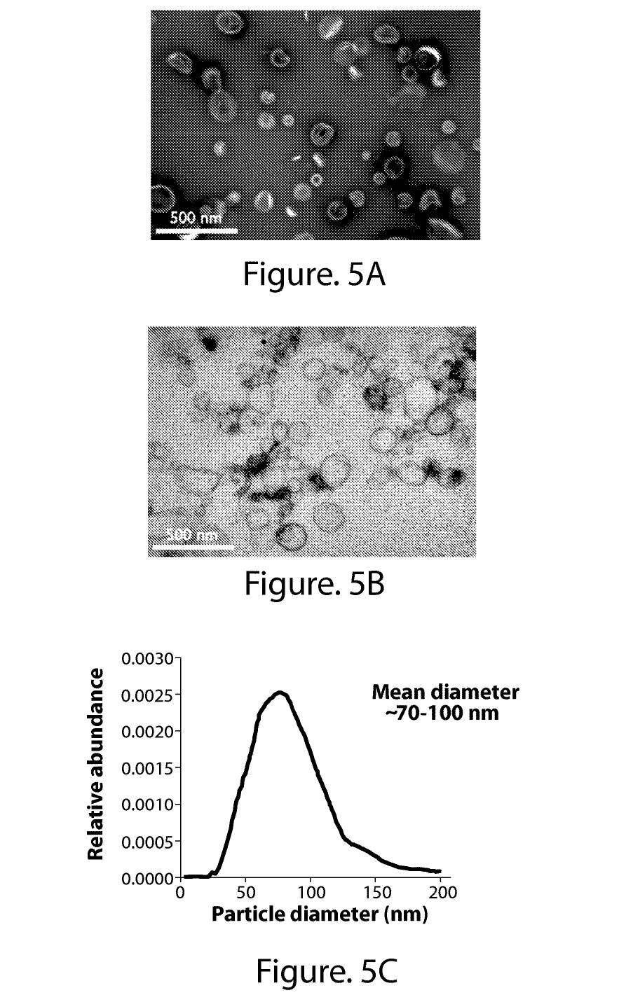

FIG. 6 demonstrates that Prochlorococcus releases vesicles continually during growth. Prochlorococcus strain MED4ax was grown under continuous light conditions, and samples were collected daily. Cell concentrations were determined by flow cytometry. Vesicles were collected by filtering 1 mL of culture through a 0.2 μm filter and measuring the total number of particles using the NanoSight instrument.

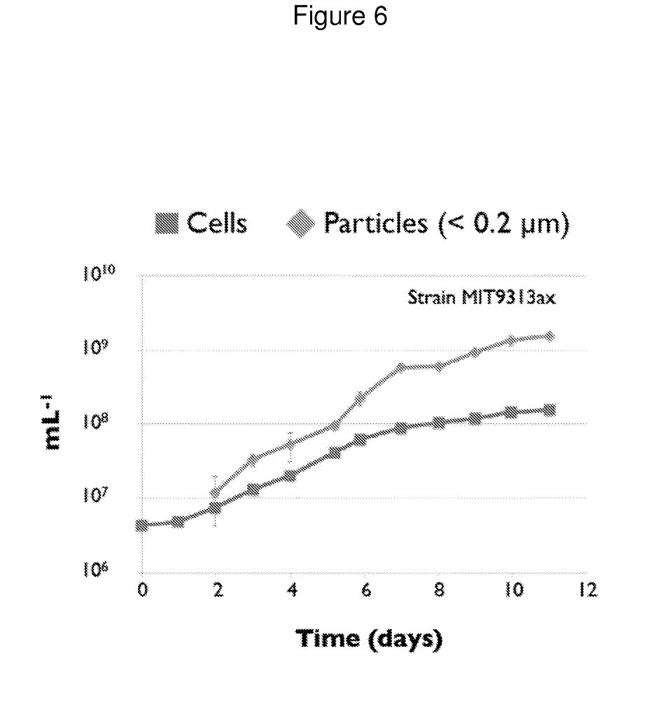

FIG. 7 demonstrates that vesicles are stable in seawater. Over the course of a week, purified vesicles held at 21 degrees C. in sterile seawater showed no significant change in (A) concentration or (B) size, as measured by the NanoSight instrument.

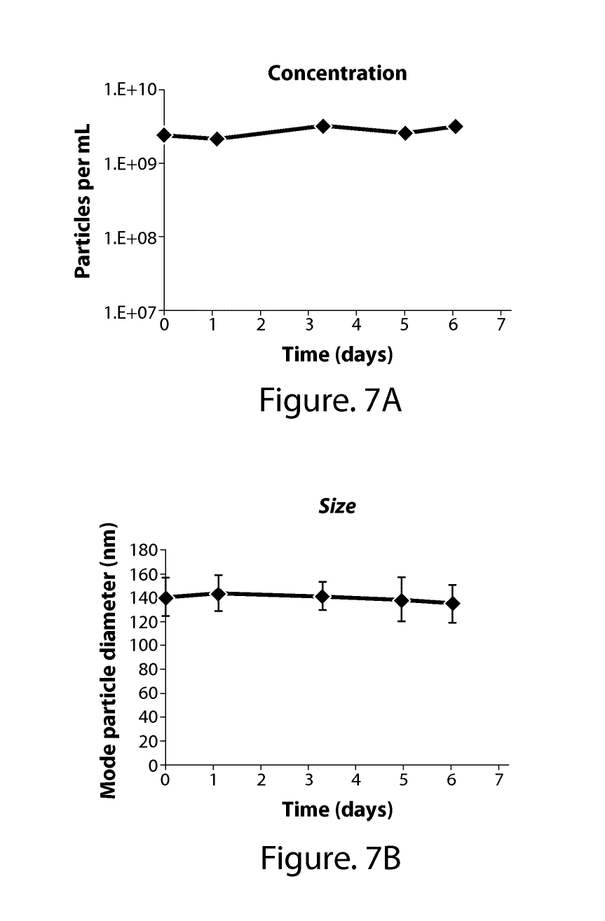

FIG. 8 demonstrates that vesicles contain lipopolysaccharides. Endotoxin, or Lipid A (A) is a standard component of bacterial outer membranes (B). The endotoxin content of purified vesicles from Prochlorococcus strain MIT9313ax (C) was measured using a commercially available kit, showing that vesicles do contain Lipid A.

FIG. 9 reveals that the vesicles also contain protein. Purified vesicle protein content was compared to whole cell lysate, as visualized on a 1-D silver stained SDS-polyacrylamide gel. Non-limiting examples of vesicle-associated proteins identified include transporters, enzymes, including proteases and sulfatases, porins and P-binding proteins.

FIG. 10 demonstrates that the vesicles also contain DNA, as measured by a standard PicoGreen fluorometric assay. Vesicles were purified from a MED4ax culture, treated with Turbo DNAse to remove contaminating DNA, and then DNA within vesicles was extracted. Prochlorococcus genomic sequences can be amplified from the purified vesicle DNA using PCR.

FIG. 11 reveals that vesicle DNA is heterogeneously sized. Vesicles were purified from a MED4ax culture, treated with Turbo DNAse to remove contaminating DNA, and then DNA within vesicles was extracted and sequenced. FIG. 11 shows a BioAnalyzer trace of vesicle-derived DNA revealing fragments of at least 4 kB.

FIG. 12 depicts sequenced reads of genomic DNA that collectively covered 40% of the genome. Vesicles from Prochlorococcus MED4ax were purified, then the DNA was isolated and sequenced. DNA sequence reads are shown mapped against the MED4 genome.

FIG. 13 reveals that Prochlorococcus vesicles also contain RNA, as determined using a fluorometric RiboGreen assay on purified vesicles. Treating the sample with RNAse reduced the RNA signal, confirming that RNA was present.

FIG. 14 reveals that vesicles are a component of natural seawater. FIG. 14A presents membrane vesicles in surface seawater collected just off the coast of Woods Hole, Mass. FIG. 14B presents vesicles from Prochlorococcus cultures for comparison. Samples were purified from seawater and visualized by transmission electron microscopy.

FIG. 15 reveals vesicles within samples collected from the oligotrophic ocean. Samples were purified from seawater and visualized by transmission electron microscopy.

### DETAILED DESCRIPTION

The invention is based, at least in part, on the surprising discovery that multiple strains of photosynthetic cyanobacteria naturally release small vesicles containing macromolecules such as lipids and proteins. This is the first observation of lipid vesicle production by a bacterium that relies solely on solar energy as an energy source, and requires no exogenous organic carbon for growth. This finding suggests a novel mechanism for production of biofuels or lipid-based chemical feedstocks, or for harvesting proteins, involving in some embodiments maintaining a population of cells, growing at a constant rate, while the vesicles are continually separated away from the cells. Since the vesicles are chemically simpler than the parent cells that secrete them, the process of breaking the vesicles open to extract lipids or proteins in some embodiments may require fewer hazardous chemicals or energy-intensive processes than are currently needed to extract lipids or proteins.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Aspects of the invention relate to photosynthetic cyanobacteria that secrete lipid-containing vesicles. It should be appreciated that any strain of photosynthetic cyanobacteria that secretes lipid-containing vesicles can be compatible with aspects of the invention. In some embodiments, the photosynthetic cyanobacterium is Prochlorococcus spp., Synechococcus spp., Synechocystis spp or Nostoc spp. For example, in non-limiting embodiments, the photosynthetic cyanobacteria can be Prochlorococcus strain MED4, Prochlorococcus strain NATL2A, Prochlorococcus strain MIT9313, Prochlorococcus strain MIT9202, Prochlorococcus strain C12Bax, Prochlorococcus strain MIT9301, Prochlorococcus strain SBax, Synechocystis PCC6803 or Synechococcus strain WH8102. In some preferred embodiments, the photosynthetic cyanobacterium is Prochlorococcus spp., members of which are efficient light absorbers and possess streamlined genomes and regulatory systems. In some embodiments, the bacterial strain is genetically modified or engineered. For example, in certain embodiments, the bacterial strain is a genetically engineered Synechococcus strain.

As used herein, “secreting vesicles,” including vesicles that contain macromolecules, such as lipid-containing vesicles and protein-containing vesicles, and the like refer(s) to a process by which a cell produces and releases a vesicle such as a lipid-containing or protein-containing vesicle. “Secreting,” “releasing” or “producing” vesicles are used interchangeably herein. Secreting a vesicle can occur by blebbing, whereby the vesicle that is secreted contains outer membrane material from the cell. Vesicles described herein can contain any type of macromolecule, non-limiting examples of which are lipids, proteins, DNA or RNA. It should be appreciated that the size of lipid-containing vesicles can vary. In some embodiments, the vesicles are less than 10 nm in diameter, or are approximately 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, or 300 nm in diameter.

Unlike previous methods for purifying lipids from photosynthetic cells, or harvesting proteins, using methods described herein, the cells do not need to be lysed in order to harvest lipid-containing vesicles or protein-containing vesicles since the vesicles are secreted from the cell. The vesicles that are secreted into the growth medium in which they are cultured, can be harvested simply by separating them from the cyanobacterial cells that secreted them and/or from the growth medium into which they were secreted. Separation of the vesicles from the cyanobacterial cells and/or growth medium can be based on movement across a filter barrier that separates vesicles from cells based on size. In some embodiments, the lipid-containing vesicles are skimmed or decanted from the growth medium. In other embodiments separation is achieved by taking advantage of potential differences in buoyancy of cells and vesicles under certain conditions, or by any other means familiar to one of ordinary skill in the art. In some embodiments, the lipid-containing vesicles are collected using techniques such as density gradient centrifugation, tangential flow filtration to concentrate vesicles, adsorption to a column based on the chemical properties of the lipids, or other approaches known to one of ordinary skill in the art.

It should be appreciated that photosynthetic cyanobacteria associated with the invention can be cultured according to any means known to one of ordinary skill in the art and in any standard vessel. For example the photosynthetic cyanobacteria can be cultured in batch or continuous growth conditions. In some embodiments, the photosynthetic cyanobacteria are cultured within a bioreactor. For example, a bioreactor can maintain a population of photosynthetic cyanobacterial cells growing in a continual (chemostat-like) growth state, with the vesicles continually separating from the bulk biomass for recovery. This stands in contrast to many existing approaches that aim to maximize bulk cellular biomass through batch growth for harvesting and extraction. In other embodiments, a dual-purpose system can be used wherein lipid yields are further increased by independently collecting both the vesicle fraction and the cells that are removed from the effluent of the continuous culture. In other embodiments, the photosynthetic cyanobacteria are cultured within an open pond system.

In some embodiments, the photosynthetic cyanobacteria and/or vesicles secreted by the photosynthetic cyanobacteria are collected from a source where the photosynthetic cyanobacterium occurs naturally such as from a body of water. In certain embodiments, the photosynthetic cyanobacteria and/or vesicles secreted by the photosynthetic cyanobacteria are collected from sea water. Following collection, the photosynthetic cyanobacteria and/or the vesicles secreted from the photosynthetic cyanobacteria are concentrated.

In some embodiments, photosynthetic cyanobacterial cells described herein can be cultured in axenic conditions, meaning that the cells are not associated with other living organisms. In such embodiments, it is not necessary to add an exogenous carbon source to the growth media containing the cells. In other embodiments, the photosynthetic cyanobacterial cells described herein can be co-cultured. In certain embodiments, the photosynthetic cyanobacterial cells described herein are co-cultured with heterotrophic cells. In certain embodiments an exogenous carbon source is added to the growth medium containing the photosynthetic cyanobacterial cells co-cultured with one or more heterotrophs. In some embodiments the cells are exposed to a constant light flux while in other embodiments, the cells are subjected to a dark/light cycle.

In some embodiments, vesicles, such as lipid-containing and/or protein-containing vesicles, are produced at a concentration of at least 10² ml⁻¹, 10³ ml⁻¹, 10⁴ ml⁻¹, 10⁵ ml⁻¹, 10⁶ ml⁻¹, 10⁷ ml⁻¹, 10⁸ ml⁻¹, 10⁹ ml⁻¹, 10¹⁰ ml⁻¹, 10¹¹ ml⁻¹, 10¹² ml⁻¹, 10¹³ ml⁻¹, 10¹⁴ ml⁻¹, 10¹⁵ ml⁻¹, 10¹⁶ ml⁻¹, 10¹⁷ ml⁻¹, 10¹⁸ ml⁻¹, 10¹⁹ ml⁻¹, 10²⁰ ml⁻¹ in growth medium. In some embodiments at least 0.05 mg lipid L⁻¹, 0.06 mg lipid L⁻¹, 0.07 mg lipid L⁻¹, 0.08 mg lipid L⁻¹, 0.09 mg lipid L⁻¹, 0.1 mg lipid L⁻¹, 0.2 mg lipid L⁻¹, 0.3 mg lipid L⁻¹, 0.4 mg lipid L⁻¹, 0.5 mg lipid L⁻¹, 0.6 mg lipid L⁻¹, 0.7 mg lipid L⁻¹, 0.8 mg lipid L⁻¹, 0.9 mg lipid L⁻¹, 1.0 mg lipid L⁻¹ or more than 1.0 mg lipid L⁻¹ are produced.

Cells according to the invention can be cultured in media of any type (rich or minimal) and composition. As would be understood by one of ordinary skill in the art, routine optimization would allow for use of a variety of types of media. The selected medium can be supplemented with various additional components. Similarly, other aspects of the medium and growth conditions may be optimized through routine experimentation. For example, pH, salinity and temperature are non-limiting examples of factors that can be optimized.

Aspects of the invention relate to a cell culture as described herein or produced by methods described herein.

Further aspects of the invention relate to a composition comprising isolated vesicles, such as lipid-containing and/or protein-containing vesicles, of photosynthetic cyanobacteria. In some embodiments, the vesicles are present at a concentration of at least 10⁷ ml⁻¹. In some embodiments, the vesicles are substantially free of cyanobacterial cells. In some embodiments, the vesicles are purified by filtration and or by density gradient centrifugation.

Methods for producing and harvesting lipids described herein offer significant advantages over most current batch process systems: they do not require separate, energy-intensive processes to collect and extract the bulk biomass in order to obtain the lipids; they are driven by solar energy so additions of organic carbon sources are not required to facilitate growth and lipid production; and by keeping cells in continuous culture, there is less waste of production days due to the need to collect the biomass and re-start a new culture.

As would be appreciated by one of ordinary skill in the art, chemical approaches, such as transesterification, can be used to convert lipids such as triacylglycerol into the alkyl esters that comprise biodiesel.

Methods and compositions described herein are useful, for example, for a novel mechanism of biodiesel/biofuel production and or for packaging of high-value, biologically-synthesized products—chemical feedstocks, pharmaceuticals or other useful natural products—into vesicles for extracellular release that facilitates easy recovery of the material.

It should be appreciated that any kind of lipid can be produced by methods described herein, including neutral or polar lipids. In some embodiments, the lipid is a triacylglyceride (TAG), relevant for biofuel production. In some embodiments, the cyanobacteria that produces a TAG is of the genus Nostoc.

It should be appreciated that any kind of protein can be produced by methods described herein. In some embodiments, a cyanobacterium is engineered such that a signal sequence is added to a protein of interest that would direct it across the cell's inner membrane, where the protein could be packaged into a vesicle and released. Vesicles can also enable collection of large amounts of membrane-bound proteins. In some embodiments, the protein is a transporter, an enzyme, such as a protease or sulfatase, a porin or a P-binding protein. This is a versatile process of protein packaging into bacterial vesicles, because the protein can be membrane-bound or not. In some embodiments, the protein is successfully translocated into the cell's periplasm and folded there. The protein can then be harvested from vesicles. In some embodiments, the protein that is produced and harvested from the cell is naturally produced by the cell while in other embodiments the protein is not naturally produced by the cell. In some embodiments, nucleic acids such as RNA or DNA are harvested from the cell. A nucleic acid harvested from a cell may or may not encode a gene.

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference.

### EXAMPLES

### Example 1

### Production of Outer Membrane Vesicles by Cells Such as Prochlorococcus Cells

The difficulties inherent in biomass recovery and lipid extraction have motivated research into systems where lipids are instead directly secreted from photosynthetic cells. To date, the work in this field has involved genetically engineering specific fatty acid synthesis and transport pathways into photosynthetic organisms of interest (9, 19). An alternate model for lipid secretion has been suggested by studies in Escherichia coli and Pseudomonas aeruginosa revealing that these heterotrophic cells naturally release lipids in the form of small vesicles (8). These vesicles, ranging in size from 20 nm-250 nm in diameter, are thought to arise from the outer membrane of these Gram-negative cells and contain both a single lipid membrane along with assorted proteins and small molecules (11). Outer membrane-derived vesicles play roles in multiple biological processes including quorum signaling, biofilm formation, DNA transfer, and pathogenesis (10, 17, 18, 20).

As described herein, it was observed that multiple Prochlorococcus strains release small “blebs” into the surrounding media. These structures appear to be lipid-enclosed membrane vesicles. Following from this discovery, provided herein is a novel mechanism for photosynthetic production of lipids in cells such as Prochlorococcus cells.

The marine cyanobacterium Prochlorococcus is the smallest and most abundant photosynthetic organism on the planet (15). Due to its small cell size and high pigment content, Prochloroccocus is also the most efficient light absorber among all microalgae (12). Furthermore, this organism has the most streamlined genome and regulatory system of any known photosynthetic organism (4).

### Methods

### Culture Conditions

Prochlorococcus strains were grown in seawater-based PRO99 media (Moore, L. et al. “Culturing the marine cyanobacterium Prochlorococcus.” Limnol. Oceanogr.: Methods 1-10. 2007) under either constant light flux (10-40 μmol Q m⁻² s⁻¹, depending on the strain) or a 13 hr light:11 hr dark cycle at 21-23° C. For growing large cultures (1 L or greater), the culture media was supplemented with 10 mM sodium bicarbonate upon inoculation. In certain experiments, Prochlorococcus strains were grown in ProMM medium (14, also known as PLAG medium). In some embodiments, culture volumes are greater than 20 L.

### Collecting Vesicles

Cultures were grown to mid-exponential, late-exponential or stationary phase. For small culture volumes (<40 mL), extracellular vesicles were separated from cyanobacterial cells by filtration through a 0.2 μm filter. For large culture volumes, the culture was filtered through a Polycap TC 0.2 μm filter (Whatman) (by gravity or <1 psi vacuum pressure) and then concentrated using an Ultrasette 100 kDa tangential flow filtration device (Pall). In some embodiments, vesicles were pelleted by ultracentrifugation at 100,000×g (2 hrs, 4° C.) to further concentrate or change buffer conditions.

Additional purification for biochemical assays was accomplished by running the vesicles through a density gradient. The vesicle pellet is resuspended in 45% Optiprep density gradient medium (Sigma, St Louis, Mo.), and overlaid with equal volumes of 40%, 35%, 30%, 25%, 20%, 15%, 10% and 0% Optiprep. This was centrifuged at 100,000×g for 6 hours, and gradient fractions were recovered. Fractions containing vesicles were identified by running a sample of each fraction on a polyacrylamide gel, staining with silver stain or Coomassie blue, and identifying the lanes with the strongest protein bands. To recover the vesicle material, the fraction or fractions containing the vesicles were combined and diluted at least 5-fold with buffer and ultracentrifuged (100,000×g, 1 hr).

### Visualization

Membrane vesicles were visualized by transmission electron microscopy using negative staining with 2% uranyl acetate.

### Enumeration of Vesicles

The concentration of vesicles found in samples was measured using a NanoSight NTA LM10HS instrument (NanoSight Ltd, UK), with concentration readings of 20-60 particles per field and additional calibration verified using Duke Scientific 3K-100 particle standards. Lipophilic dyes can be used for quantification of vesicles. Non-limiting examples of lipophilic dyes that have been tested include PKH67 (Phanos Technologies, Sigma-Aldrich) and FM4-64 (Life Technologies, Carlsbad, Calif.). In other embodiments, a DiO lipophilic tracer is used (Life Technologies, Carlsbad, Calif.). In other embodiments, an antibody is used to detect vesicles. In certain embodiments, the antibody specifically binds to lipid A, such as to a core region of lipid A. In other embodiments, a general protein dye is used to quantify vesicles, such as NanoOrange® (Life Technologies, Carlsbad, Calif.).

### Lipid Content

Production rates from cultures of axenic Prochlorococcus strains grown under normal laboratory conditions were measured. Lipid vesicles were observed at concentrations of ˜10⁹ ml⁻¹ in Prochlorococcus cultures, varying depending on the strain and growth conditions. Rates of production are examined, and lipid composition and amounts are determined. Assuming a spherical vesicle with an average diameter of 100 nm and a lipid bilayer membrane, each vesicle is estimated to contain at least ˜1.2×10⁻¹⁶ g lipid, or 0.1 mg lipid L⁻¹.

### Results

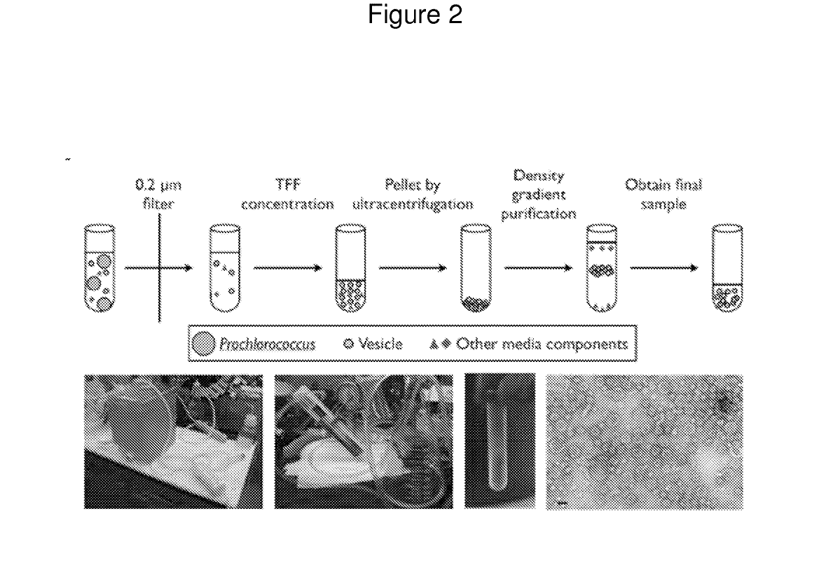

As shown in FIG. 2, a process has been developed that allows for the purification of vesicles from cultures of photosynthetic cells such as Prochlorococcus cells. Vesicles were first separated away from Prochlorococcus cells by passing the culture gently through a 0.2 μm filter. The filtered supernatant was then concentrated down to a volume of ˜40 mL using a tangential flow filter, and vesicles were pelleted by ultracentrifugation. The vesicles can be further purified through a density gradient. Vesicles are visualized by electron microscopy. Approaches such as the NanoSight NTA system (NanoSight technologies, UK), Izon qNano and Influx can make rapid and direct measurements of vesicle concentrations in cultures.

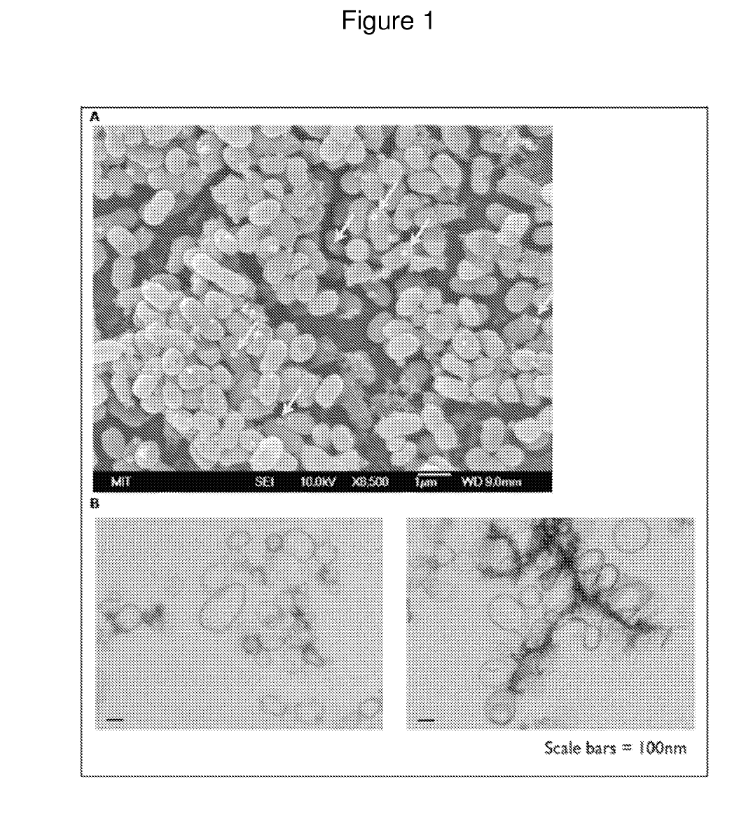

Electron micrographs (FIG. 1) of axenic Prochlorococcus cultures show the release of small (˜100 nm diameter) round structures that appear to be vesicles bound by a single lipid membrane. Vesicle production was observed at least in axenic Prochlorococcus strains MED4ax, MIT9313ax and NATL2Aax. The axenic Synechococcus strain, WH8012ax, also exhibits vesicle-sized particles in the supernatant as measured with the NanoSight instrument. Thus, vesicle production appears to occur in multiple strains of photosynthetic marine cyanobacteria.

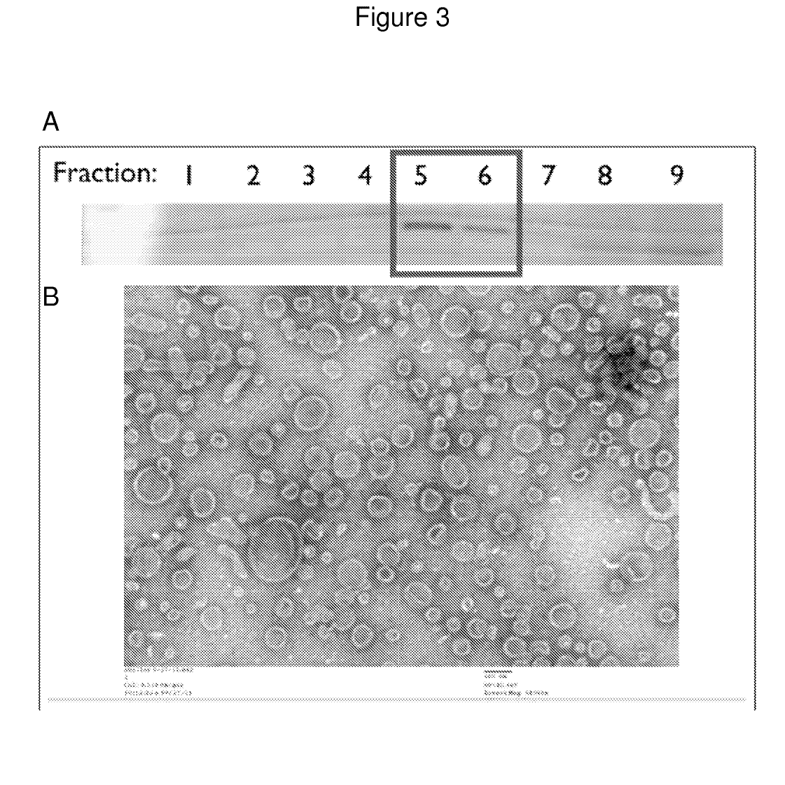

Vesicle purification from cultures of volumes such as 20 L, begins by gently filtering mid- or late-exponential phase cultures through a 0.2 μm filter (a Whatman filter capsule). The samples are then concentrated through a Pall TFF module (70 or 100 kDa cutoff) to ˜40 mL or less, and then vesicles are pelleted by ultracentrifugation. Additional purification is accomplished through buffer washes and density gradient centrifugation through an Optiprep gradient (FIG. 3). In some embodiments, the vesicles are labeled fluorescently, such as using a lipophilic dye, for example PKH67 or FM4-64.

### Example 2

### Optimization of Vesicle Production in Prochlorococcus and Synechococcus

Investigation is conducted into which strains of Prochlorococcus and Synechococcus produce lipid vesicles and whether production varies significantly among different strains. Prochlorococcus cells isolated from the upper regions of the ocean have significant differences in both genomic content and physiology from those isolated from deeper depths, so vesicle production could co-vary with some of these traits. Vesicle production is also investigated in Synechocystis sp. PCC6803, a cyanobacterium widely used in biofuel research. Initial measurements of vesicle size and concentration are made using the NanoSight, and corroborated with standard scanning and transmission electron microscopy techniques. A systematic survey of vesicle production is conducted in one or a few selected strains under a variety of conditions. Using the NanoSight, vesicle production is followed across a complete growth curve, from lag phase to mid-exponential and stationary phase, to see if rates of lipid release vary. How vesicle production might vary in cultures grown under constant irradiance as opposed to a typical day-night cycle, at different salinities, temperatures, and under various stress conditions is also investigated. Together these studies elucidate the physiological variables that influence vesicle production, useful information for maximizing lipid production in a biofuel production context.

### Example 3

### Characterization of the Content of Vesicles

Vesicle production has now been demonstrated from strains MED4ax, NATL2Aax and 9313ax growing under continuous light conditions. Small particles in the culture were found to increase in concentration continually during exponential cell growth, and in some cases appear to increase even further during stationary phase. On average, vesicle content in exponentially-growing cells averages 10⁸/mL. The small particles were observed to be stable over the course of several days.

### Lipid

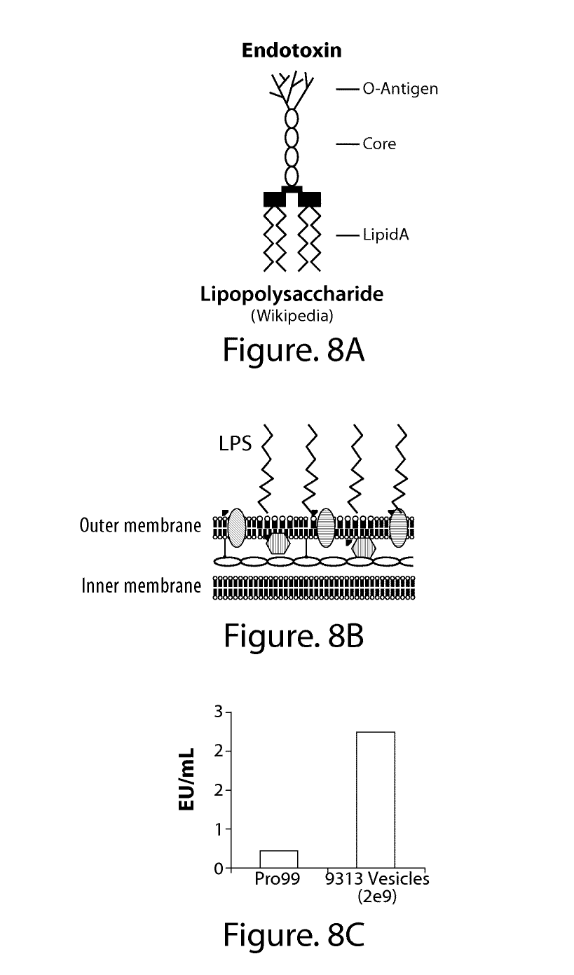

FIG. 8 demonstrates that vesicles contain lipopolysaccharides. To assess the potential of lipid vesicle-derived material as a source of biofuels or other valuable natural products, the composition of these structures from both biological and chemical perspectives were determined. The identity and amount of fatty acids contained in the vesicles were determined using HPLC-MS/HPLC-QToF-MS approaches. These analyses allow for a comparison of the lipid profiles of vesicles vs. the whole cells and identify the lipid species released. The presence of Lipid A (endotoxin) in the vesicles has been confirmed, suggesting that the vesicles are derived from outer membranes.

### Protein

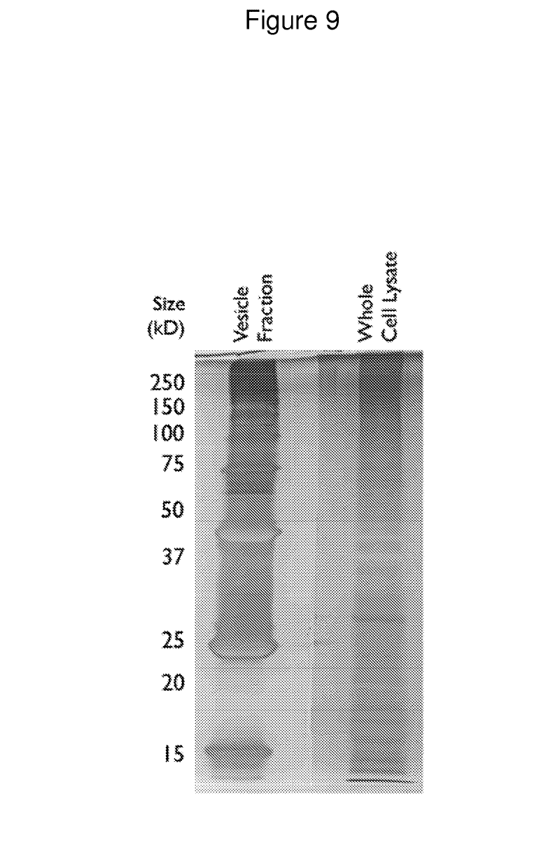

Preliminary experiments indicated that the released vesicles also contain protein. Proteins from purified vesicles were separated on standard 1-D polyacrylamide gels and then identified by mass spectrometry (FIG. 9). The identity and relative abundance of proteins in the vesicles is compared to the proteome of the putative origin of these vesicles—the Prochlorococcus outer membrane—to see if the vesicles are enriched in any specific set of proteins. This information provide clues as to how proteins are packaged into lipid vesicles, which can be exploited for packaging specific proteins of interest for vesicle secretion.

By concentrating vesicles from ˜2 L of culture, sufficient biomass was obtained to begin characterizing the proteome of vesicles produced by strain MIT9313ax. A vesicle sample was run out on a 1D gel, silver stained, and major bands were identified by mass spectrometry. Many of the high-confidence identifications were for proteins predicted to be membrane-bound and have a non-cytoplasmic cellular localization, consistent with these vesicles being derived from the outer membrane. Protein data has also been obtained for strain MED4ax. Many of the identified proteins have no known function.

### DNA

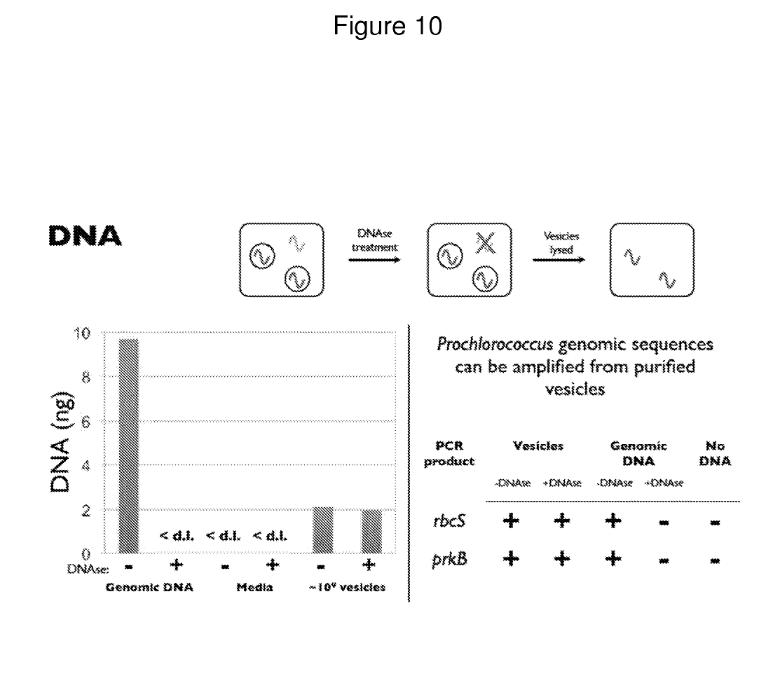

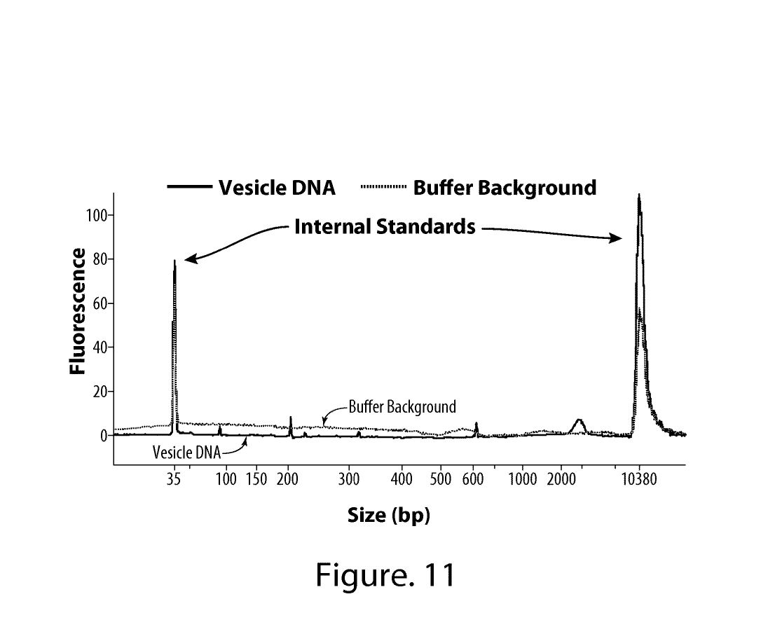

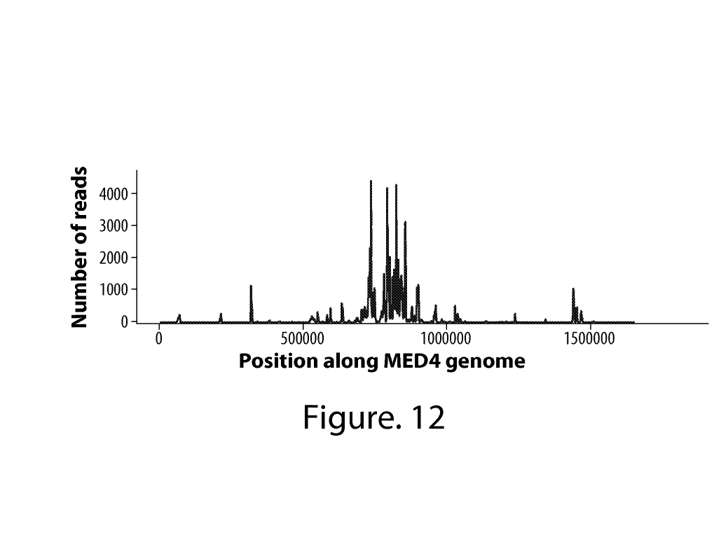

DNA is found in at least some of the vesicles (FIGS. 10-12). Purified, enriched vesicles were treated with Turbo DNAse (Life Technologies, Carlsbad, Calif.) to remove any remaining extracellular contaminating DNA, washed, and then lysed. DNA content was measured by a PicoGreen assay (Life Technologies, Carlsbad, Calif.) and a clear enrichment of DNA above background was found in the sample. The distribution appears to be heterogenous, with some vesicles having large fragments and others carrying only short pieces. Fragments of at least 4 kB were detected, more than sufficient in size to potentially carry functional genes.

To verify the DNA content of vesicles, vesicle contents were amplified by multiple displacement amplification for sequencing. By pooling multiple independent amplification reactions, libraries were generated for strain MED4ax. This confirmed that the DNA was in fact MED4 genomic sequences; ˜40% of the total genome was able to be recovered from within the vesicles. The distribution of reads was skewed toward the middle of the genome. Without wishing to be bound by any theory, this could be due to an MDA artifact (even though independent reactions were pooled to minimize the chance of ‘jackpot’ amplifications) or could represent a biologically relevant event.

### RNA

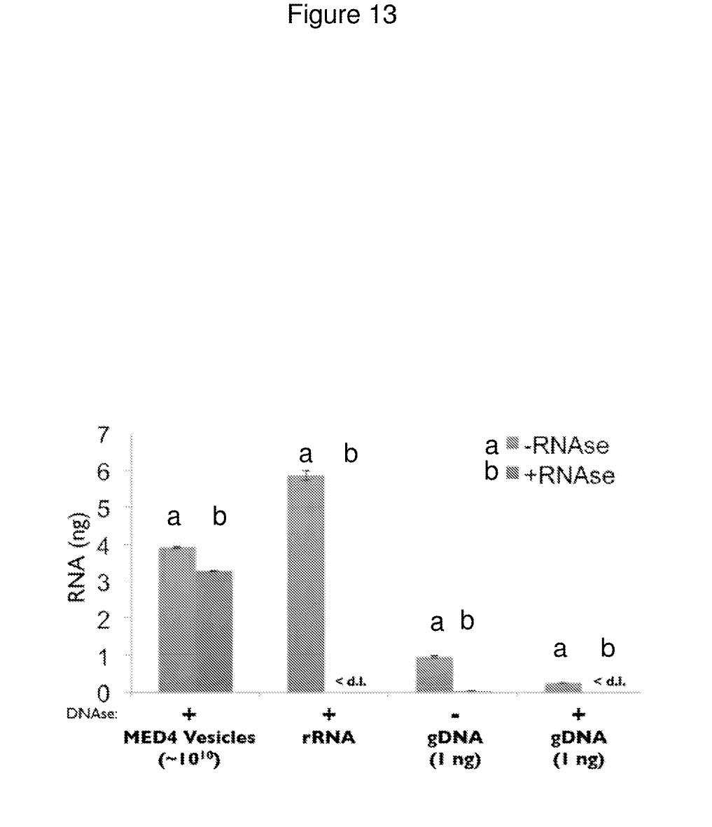

RNA was also found to be associated with the vesicles (FIG. 13). The RNA was isolated using RNAclean XP beads from a lysed vesicle sample and treated with Turbo DNAse to remove any contaminating DNA, then prepared as a RNAseq library but without any ribosomal RNA subtraction. RNA was also detected by a RiboGreen® (Life Technologies, Carlsbad, Calif.) assay.

### Example 4

### Evaluation of Co-Culturing Conditions to Improve Vesicle Production by Phototrophs

The potential benefits of co-culturing conditions to increase yields and improve the long-term stability of biofuel-producing bioreactors have been examined previously (1, 3). Co-cultures of different microbes can improve productivity in many ways, including stabilizing culture conditions, reducing the buildup of toxic byproducts, through the exchange of critical signaling compounds, or taking advantage of synergistic metabolic pathways found in the two strains (1). Any potential use of cyanobacteria for biofuel production will require a better understanding of conditions facilitating large-scale culturing.

Prochlorococcus provides a useful model for studying these processes in the context of maximizing lipid vesicle output. Laboratory cultures of Prochlorococcus are already known to grow more rapidly in the presence of heterotrophic bacteria (21); the advantage to Prochlorococcus comes from the heterotrophs' ability to mitigate the toxicity of reactive oxygen species (13, 14), but the carbon sources used by the heterotrophs in co-culture conditions are unknown. Heterotroph growth under coculture conditions may be, at least in part, facilitated by the lipid vesicles serving as a source of fixed carbon. Without wishing to be bound by any theory, if heterotrophs are used to stabilize mass cultures, and they do consume vesicles, then the beneficial effects on growth of their photosynthetic partner could be mitigated by vesicle consumption. To determine if the presence of heterotrophs influences vesicle production, it is first established whether heterotrophs themselves release lipid vesicles and, if they do, whether they can be readily distinguished from Prochlorococcus vesicles. NanoSight is used to characterize vesicle production from a set of diverse heterotroph strains that all enhance Prochlorococcus growth (21). Next, a series of co-cultures are grown—each containing Prochlorococcus, one heterotroph, and no added organic carbon source—and the total number of vesicles produced is measured. Prochlorococcus growth and vesicle production rates in co-culture are compared to those found in pure cultures. If the heterotrophs themselves do produce lipid vesicles, the relative impact of heterotroph-produced vesicles to cyanobacterial vesicles on total lipid output is measured.

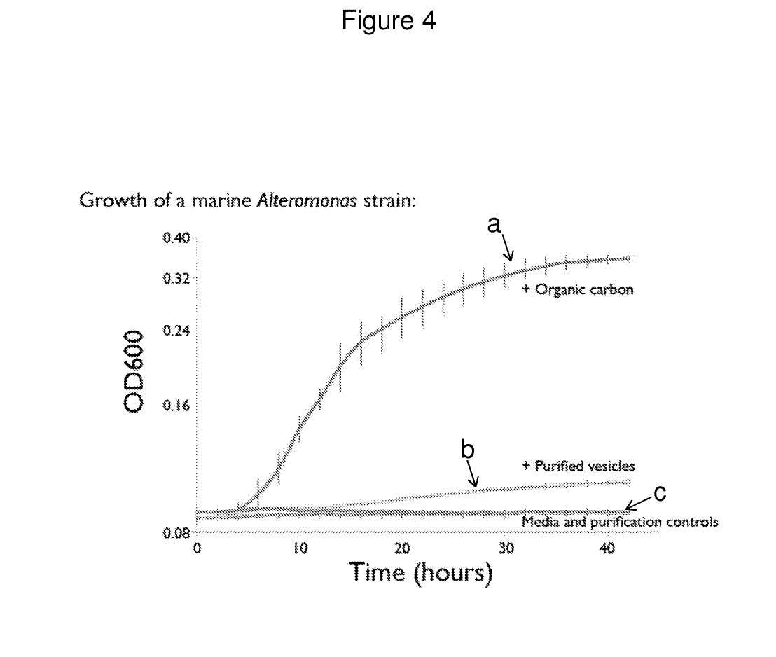

To determine whether heterotrophic organisms living in proximity to Prochlorococcus might be able to use vesicles as a carbon source, a heterotroph strain was grown in the presence of purified, enriched vesicles (both with and without any other carbon source) (FIG. 4). Vesicles were able to function as the carbon source for three heterotroph strains tested (not all shown in FIG. 4); addition of vesicles both allowed growth in Pro99 (where they otherwise were not be able to grow) and also affected the growth dynamics in a medium (ProMM) that already contained carbon sources. Some tested strains did not grow in the presence of the vesicles, arguing that the vesicle preparation was not overly contaminated with other carbon sources or other heterotrophic bacteria.

To see if heterotrophs can consume lipid vesicles, it is investigated whether heterotrophs will grow in media containing purified vesicles as the sole carbon source. If this is the case, then culture conditions are investigated wherein the cyanobacteria could still be stabilized by the presence of heterotrophs while vesicle loss is minimized. For example, it is investigated whether adding small amounts of an alternate organic carbon source (such as glucose, pyruvate, or glycerol) can support heterotroph growth while preventing vesicle degradation by providing a more easily utilized energy source.

### Example 5

### Isolation of Vesicles from Natural Marine Ecosystems

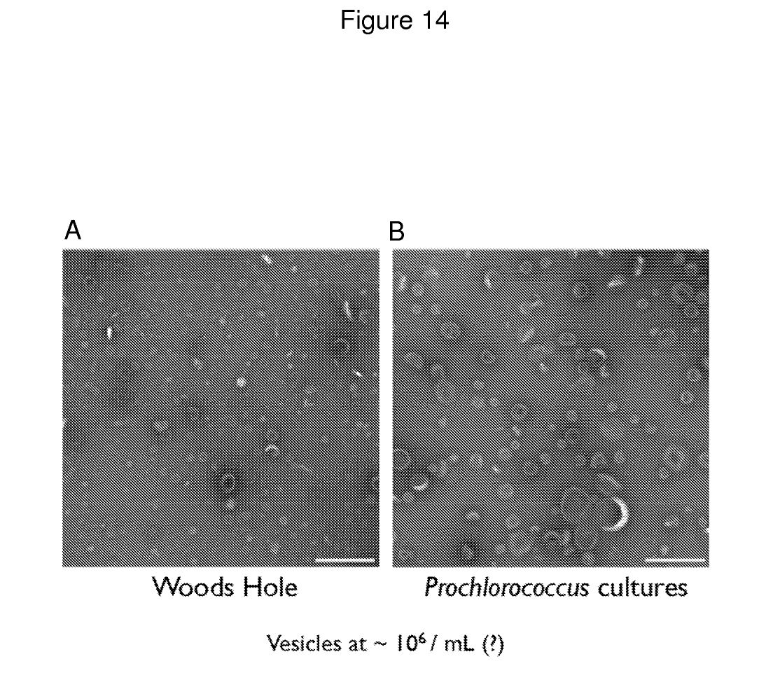

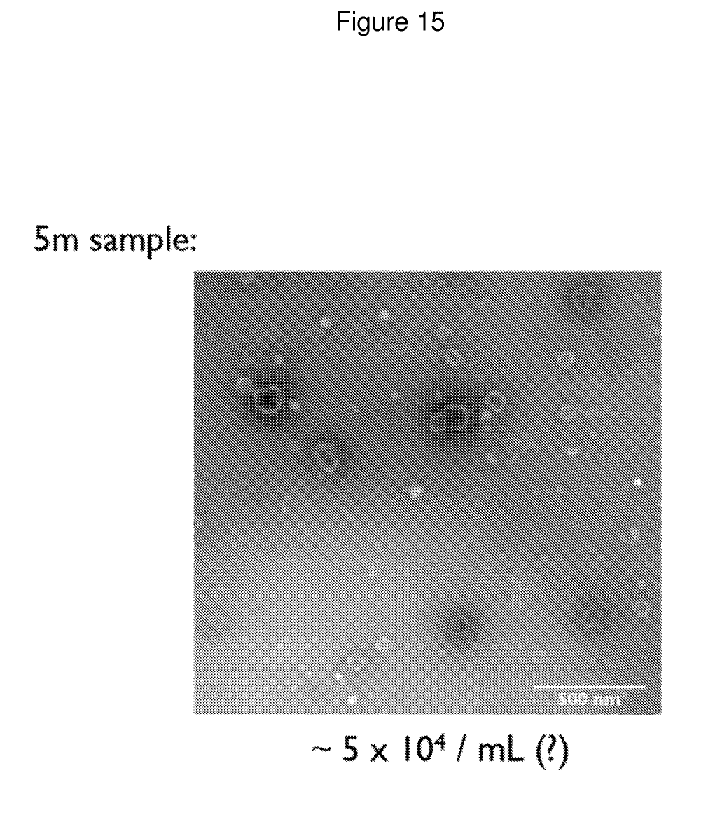

Particles were isolated from ˜100 L of surface water at Woods Hole, Mass. By TEM, the particles resembled membrane vesicles (FIG. 14). To expand the understanding of the abundance and distribution of membrane vesicles in the ocean, a set of samples was collected from Bermuda. Several depths were sampled including 5 m (surface) (FIG. 15), 70 m (deep chlorophyll maximum), 150 m (low euphotic zone) and 500 m (‘deep’ sample). For each sample, 280 L seawater was collected and concentrated for vesicles. The bulk of the water was concentrated using a Pall Centramate tangential flow filter with 5 100 kDa filters. Three replicates of 25 L were collected and used for metagenomics. Also collected were flow cytometry samples and total in situ small particles. These samples are used to characterize the presence of vesicles, estimate their in situ concentrations, and sequence their DNA and/or RNA to examine the diversity of vesicle producers and/or the extent of the genetic reservoir that vesicles represent.

### REFERENCES

1. Bader, J., E. Mast-Gerlach, M. K. Popović, R. Bajpai, and U. Stahl. 2009. Relevance of microbial coculture fermentations in biotechnology. Journal of Applied Microbiology 109:371-387.
2. Chisti, Y. 2008. Response to Reijnders: Do biofuels from microalgae beat biofuels from terrestrial plants? Trends in Biotechnology 26:351-352.
3. Christenson, L., and R. Sims. 2011. Production and harvesting of microalgae for wastewater treatment, biofuels, and bioproducts. Biotechnology Advances. Elsevier Inc. 29:686-702.
4. Coleman, M. L., and S. W. Chisholm. 2007. Code and context: Prochlorococcus as a model for cross-scale biology. Trends Microbiol 15:398-407.
5. Hannon, M., J. Gimpel, M. Tran, B. Rasala, and S. Mayfield. 2010. Biofuels from algae: challenges and potential. Biofuels 1:763-784.
6. Hu, Q., M. Sommerfeld, E. Jarvis, M. Ghirardi, M. Posewitz, M. Seibert, and A. Darzins. 2008. Microalgal triacylglycerols as feedstocks for biofuel production: perspectives and advances. Plant J. 54:621-639.
7. Knoshaug, E., and A. Darzins. 2011. Algal Biofuels: THE PROCESS. Chemical Engineering Progress 107:37-47.
8. Kulp, A., and M. J. Kuehn. 2010. Biological functions and biogenesis of secreted bacterial outer membrane vesicles. Annu Rev Microbiol 64:163-184.
9. Liu, X., J. Sheng, and R. Curtiss. 2011. Fatty acid production in genetically modified cyanobacteria. Proceedings of the National Academy of Sciences 108:6899-6904.
10. Mashburn, L. M., and M. Whiteley. 2005. Membrane vesicles traffic signals and facilitate group activities in a prokaryote. Nature 437:422-425.
11. Mashburn-Warren, L. M., and M. Whiteley. 2006. Special delivery: vesicle trafficking in prokaryotes. Molecular Microbiology 61:839-846.
12. Morel, A., Y. Ahn, F. Partensky, D. Vaulot, and H. Claustre. 1993. Prochlorococcus and Synechococcus—a Comparative-Study of Their Optical-Properties in Relation to Their Size and Pigmentation. J Mar Res 51:617-649.
13. Morris, J. J., Z. I. Johnson, M. J. Szul, M. Keller, and E. R. Zinser. 2011. Dependence of the Cyanobacterium Prochlorococcus on Hydrogen Peroxide Scavenging Microbes for Growth at the Ocean's Surface. PLoS ONE 6:e16805.
14. Morris, J. J., R. Kirkegaard, M. J. Szul, Z. I. Johnson, and E. R. Zinser. 2008. Facilitation of robust growth of Prochlorococcus colonies and dilute liquid cultures by “helper” heterotrophic bacteria. Appl Environ Microbiol 74:4530-4534.
15. Partensky, F., W. R. Hess, and D. Vaulot. 1999. Prochlorococcus, a marine photosynthetic prokaryote of global significance. Microbiol Mol Biol Rev 63:106-127.
16. Pienkos, P. T., and A. Darzins. 2009. The promise and challenges of microalgal-derived biofuels. Biofuels, Bioprod. Bioref. 3:431-440.
17. Renelli, M., V. Matias, R. Y. Lo, and T. J. Beveridge. 2004. DNA-containing membrane vesicles of Pseudomonas aeruginosa PAO1 and their genetic transformation potential. Microbiology (Reading, Engl) 150:2161-2169.
18. Rivera, J., R. J. B. Cordero, A. S. Nakouzi, S. Frases, A. Nicola, and A. Casadevall. 2010. Bacillus anthracis produces membrane-derived vesicles containing biologically active toxins. Proceedings of the National Academy of Sciences 107:19002-19007.
19. Robertson, D. E., S. A. Jacobson, F. Morgan, D. Berry, G. M. Church, and N. B. Afeyan. 2011. A new dawn for industrial photosynthesis. Photosyn Res 107:269-277.
20. Schooling, S. R., A. Hubley, and T. J. Beveridge. 2009. Interactions of DNA with biofilm derived membrane vesicles. J Bacteriol 191:4097-4102.
21. Sher, D., J. W. Thompson, N. Kashtan, L. Croal, and S. W. Chisholm. 2011. Response of Prochlorococcus ecotypes to co-culture with diverse marine bacteria. ISME J 5:1125-1132.

### EQUIVALENTS

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Those skilled in the art will recognize, or be able to ascertain using no more than routine experimentation, many equivalents to the specific embodiments of the invention described herein. Such equivalents are intended to be encompassed by the following claims. All references, including patent documents, disclosed herein are incorporated by reference in their entirety, particularly for the disclosure referenced herein.

What is claimed is:

1. A method for harvesting lipids, comprising:
culturing in growth medium a photosynthetic cyanobacteria that secretes lipid-containing vesicles into the growth medium; and
separating the secreted lipid-containing vesicles from the cyanobacteria and/or from the growth medium.

2. The method of claim 1 further comprising collecting the lipid-containing vesicles.

3. The method of claim 1 wherein the photosynthetic cyanobacteria is of the genus Prochlorococcus, Synechococcus, Synechocystis or Nostoc.

4. The method of claim 1 wherein the photosynthetic cyanobacteria is cultured within a bioreactor or open pond system.

5. The method of claim 1 wherein a carbon source is not added to the growth medium.

6. The method of claim 1 wherein the cyanobacteria is co-cultured with a heterotrophic bacteria.

7. The method of claim 1 wherein lipid-containing vesicles are produced at a concentration of at least 10⁷ ml⁻¹ in the growth medium.

8. The method of claim 1 wherein the cyanobacterial cell is not lysed during separation of the secreted lipid-containing vesicles from the cyanobacteria.

9. The method of claim 1 wherein the photosynthetic cyanobacteria is exposed to constant light flux or to a light-dark cycle.

10. The method of claim 1 wherein the lipid-containing vesicles are purified by filtration, density gradient centrifugation or binding to an affinity column/resin.

11. The method of claim 1 wherein a population of photosynthetic cyanobacteria is kept within a continual growth state and wherein lipid-containing vesicles are continually separated from the cyanobacteria and/or from the growth medium.

12. The method of claim 1 wherein at least 0.1 mg lipid L⁻¹ are produced.

13. A cell culture produced by the method of claim 1.

14. A composition comprising isolated lipid-containing vesicles of photosynthetic cyanobacteria.

15. The composition of claim 14 wherein the lipid-containing vesicles are present at a concentration of at least 10⁷ ml⁻¹.

16. The composition of claim 14 wherein the lipid-containing vesicles are substantially free of cyanobacterial cells.

17. The composition of claim 14 wherein the lipid-containing vesicles are purified by filtration, density gradient centrifugation or binding to an affinity column/resin.

18. A method for harvesting proteins, comprising:
culturing in growth medium a photosynthetic cyanobacteria that secretes protein-containing vesicles into the growth medium; and
separating the secreted protein-containing vesicles from the cyanobacteria and/or from the growth medium.

19. The method of claim 18 further comprising collecting the protein-containing vesicles.

20. The method of claim 18 wherein the photosynthetic cyanobacteria is of the genus Prochlorococcus, Synechococcus, Synechocystis or Nostoc.

21. The method of claim 18 wherein the photosynthetic cyanobacteria is cultured within a bioreactor or open pond system.

22. The method of claim 18 wherein a carbon source is not added to the growth medium.

23. The method of claim 18 wherein the cyanobacteria is co-cultured with a heterotrophic bacteria.

24. The method of claim 18 wherein protein-containing vesicles are produced at a concentration of at least 10⁷ ml⁻¹ in the growth medium.

25. The method of claim 18 wherein the cyanobacterial cell is not lysed during separation of the secreted lipid-containing vesicles from the cyanobacteria.

26. The method of claim 18 wherein the photosynthetic cyanobacteria is exposed to constant light flux or to a light-dark cycle.

27. The method of claim 18 wherein the protein-containing vesicles are purified by filtration, density gradient centrifugation or binding to an affinity column/resin.

28. The method of claim 18 wherein a population of photosynthetic cyanobacteria is kept within a continual growth state and wherein lipid-containing vesicles are continually separated from the cyanobacteria and/or from the growth medium.

29. The method of claim 18 wherein at least 0.1 mg protein L⁻¹ produced.

30. A cell culture produced by the method of claim 18.

31. A composition comprising isolated protein-containing vesicles of photosynthetic cyanobacteria.

32. The composition of claim 31 wherein the protein-containing vesicles are present at a concentration of at least 10⁷ ml⁻¹.

33. The composition of claim 31 wherein the protein-containing vesicles are substantially free of cyanobacterial cells.

34. The composition of claim 31 wherein the protein-containing vesicles are purified by filtration, density gradient centrifugation or binding to an affinity column/resin.

35. A method for harvesting photosynthetically derived macromolecules, comprising:
concentrating a solution containing photosynthetic cyanobacteria that secrete vesicles containing photosynthetically derived macromolecules into the solution; and
separating the secreted vesicles containing photosynthetically derived macromolecules from the cyanobacteria and/or from the solution.

36. The method of claim 35 wherein the macromolecule is a protein, a nucleic acid or a lipid.
